SEQUENCE LISTING (14 sequences)

SEQ ID NO: 1 - protein, 5 aa, Artificial Sequence
  VH, CDR1
GYNMN

SEQ ID NO: 2 - protein, 17 aa, Artificial Sequence
  VH, CDR2
NIDPYYGDTDYDQKFKG

SEQ ID NO: 3 - protein, 11 aa, Artificial Sequence
  VH, CDR3
GAHRDYYAMDY

SEQ ID NO: 4 - protein, 11 aa, Artificial Sequence
  VL, CDR1
RTSENIYIYLA

SEQ ID NO: 5 - protein, 7 aa, Artificial Sequence
  VL, CDR2
NAKTLAE

SEQ ID NO: 6 - protein, 9 aa, Artificial Sequence
  VL, CDR3
QQHYGTPYT

SEQ ID NO: 7 - protein, 120 aa, Artificial Sequence
  D2C7 VH
EVHLQQSGPELEKPGASVKISCKASGYSFTGYNMNWVKQSNGKCLEWIGNIDPYYGDTDYDQKFKGKATLTADKSSNTVYMQLQSLTSEDSAVYYCARGAHRDYYAMDYWGQGTSVTVSS

SEQ ID NO: 8 - protein, 15 aa, Artificial Sequence
  amino acid linker
GGGGSGGGGSGGGGS

SEQ ID NO: 9 - protein, 107 aa, Artificial Sequence
  D2C7 VL
DIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGCGTKLEKK

SEQ ID NO: 10 - protein, 351 aa, Artificial Sequence
  PE38KDEL
KASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 11 - protein, 593 aa, Artificial Sequence
  D2C7 -(scdsFv)-PE38KDEL
EVHLQQSGPELEKPGASVKISCKASGYSFTGYNMNWVKQSNGKCLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGCGTKLEKKKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 12 - DNA, 1779 bp, Artificial Sequence
  D2C7-(scdsFv)-PE38KDEL
gaggtccacctgcagcagtctggacctgagctggagaagcctggcgcttcagtgaagatatcctgcaaggcttctggttactcattcactggctacaacatgaactgggtgaagcagagcaatggcaagtgccttgagtggattggaaatattgatccttactatggtgatactgactacgaccagaagttcaagggcaaggccacattgactgcagacaaatcctccaacacagtctacatgcagctccagagcctgacatctgaggactctgcagtctattactgtgcaagaggggcccatagggattactatgctatggactactggggtcaagggacctcagtcaccgtctcctcaggtggtggcggttcaggcggaggtggctctggcggtggcggatcggacatccagatgactcagtctccagcctccctatctgcatctgtgggagaaactgtcaccatcacatgtcgaacaagtgagaatatttacatttatttagcatggtatcagcagaaacagggaaaatctcctcagctcctggtctataatgcaaaaaccttagcagaaggtgtgccatcaaggttcagtggcagtgggtcaggcacacagttttctctgaagatcaacggcctgcagcctgaagattttgggggttattactgtcaacagcattatggcactccgtacacgttcggatgcgggaccaagctggaaaaaaaaaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaagcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaagacgagctc

SEQ ID NO: 13 - DNA, 726 bp, Artificial Sequence
  D2C7-(scdsFv)
gaggtccacctgcagcagtctggacctgagctggagaagcctggcgcttcagtgaagatatcctgcaaggcttctggttactcattcactggctacaacatgaactgggtgaagcagagcaatggcaagtgccttgagtggattggaaatattgatccttactatggtgatactgactacgaccagaagttcaagggcaaggccacattgactgcagacaaatcctccaacacagtctacatgcagctccagagcctgacatctgaggactctgcagtctattactgtgcaagaggggcccatagggattactatgctatggactactggggtcaagggacctcagtcaccgtctcctcaggtggtggcggttcaggcggaggtggctctggcggtggcggatcggacatccagatgactcagtctccagcctccctatctgcatctgtgggagaaactgtcaccatcacatgtcgaacaagtgagaatatttacatttatttagcatggtatcagcagaaacagggaaaatctcctcagctcctggtctataatgcaaaaaccttagcagaaggtgtgccatcaaggttcagtggcagtgggtcaggcacacagttttctctgaagatcaacggcctgcagcctgaagattttgggggttattactgtcaacagcattatggcactccgtacacgttcggatgcgggaccaagctggaaaaaaaa

SEQ ID NO: 14 - DNA, 1053 bp, Artificial Sequence
  PE38KDEL
aaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaagcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaagacgagctc

(19) United States
(12) Patent Application Publication
(10) Pub. No.: US 2021/0214442 A1
(43) Pub. Date: Jul. 15, 2021

(54) NEOADJUVANT CANCER TREATMENT WITH IMMUNOTOXIN AND CHECKPOINT INHIBITOR COMBINATION

(71) Applicant: DUKE UNIVERSITY, Durham, NC (US)

(72) Inventors: Darell BIGNER, Mebane, NC (US); Smita NAIR, Cary, NC (US); Vidyalakshmi CHANDRAMOHAN, Durham, NC (US)

(73) Assignee: DUKE UNIVERSITY, Durham, NC (US)

(21) Appl. No.: 17/055,639

(22) PCT Filed: May 16, 2019
(86) PCT No.: PCT/US19/32671

§ 371 Date: Nov. 16, 2020

Related U.S. Application Data
(60) Provisional application No. 62/672,150, filed on May 16, 2018.
(60) Provisional application No. 62/675,263, filed on May 23, 2018.
(60) Provisional application No. 62/844,857, filed on May 8, 2019.

(51) Int. Cl. C07K 16/28; C07K 16/24; A61P 35/00

(52) U.S. Cl. CPC C07K 16/2818; C07K 16/2827; A61K 2039/505; A61P 35/00; C07K 16/243

(57) ABSTRACT

Provided is a method of treating a tumor in an individual by neoadjuvant therapy, wherein the individual has not previously undergone a resection of the tumor, the method comprising administering an immunotoxin alone or an immune checkpoint inhibitor and an immunotoxin, such as D2C7-immunotoxin (D2C7-IT), followed by resection of the tumor. The method may further comprise administration of immune checkpoint inhibitor following resection.

### CROSS-REFERENCE TO RELATED APPLICATIONS

This application claims priority to U.S. Provisional Application Nos. 62/672,150 filed May 16, 2018, 62/675,263 filed May 23, 2018 and 62/844,857 filed May 8, 2019, the contents of each are incorporated by reference in their entireties.

### STATEMENT REGARDING FEDERALLY SPONSORED RESEARCH OR DEVELOPMENT

This invention was made with government support under Federal Grant No.: CA197264 awarded by the National Institutes of Health. The government has certain rights in the invention.

### TECHNICAL FIELD OF THE INVENTION

This invention is related to the area of anti-tumor immunotherapy. In particular, it relates to cancer treatment with an immunotoxin by itself or in combination with an immune checkpoint inhibitor in a neoadjuvant therapy.

### BACKGROUND OF THE INVENTION

Glioblastoma is the most dismal malignant brain tumor among all primary brain and central nervous system tumors. The median survival time for glioblastoma patients with the current standard treatment or even newly developed agents is less than 15 months. Thus, there is an urgent need to develop advanced and efficient therapeutic approaches to improve the poor survival outlook of glioblastoma patients as well as other tumors expressing EGFR receptors.

### SUMMARY OF THE INVENTION

According to one aspect of the invention a method of treating a tumor in an individual by neoadjuvant therapy is provided. In this method, the individual has not previously undergone a resection to treat the tumor (e.g., no surgical treatment to reduce tumor burden). The method of treating a tumor in an individual by neoadjuvant therapy comprises administering an effective amount of an immunotoxin or of an immunotoxin and immune checkpoint inhibitor, wherein the immunotoxin comprises an antibody or antigen binding region thereof, suitably a single chain variable region antibody, fused to a PE38 truncated Pseudomonas exotoxin, and then the individual is treated to reduce tumor burden. In combination therapy, immunotoxin and immune checkpoint inhibitor may be administered to the individual, either at the same time or sequentially in relation. Tumor burden may be reduced such as by surgical resection. Such resection of tumor can occur in a time period ranging from 2 weeks to a few months following administration of an immune checkpoint inhibitor and the immunotoxin. The immunotoxin comprises antibody or antigen binding region thereof, such as a single chain variable region antibody, fused to a PE38 truncated Pseudomonas exotoxin, wherein the single chain variable region antibody has CDR1, CDR2, and CDR3 regions as shown in SEQ ID NO: 1-6 (“D2C7-IT”) or comprises an antigen binding fragment thereof.

According to another aspect of the invention, provided is a method for neoadjuvant immunotherapy of cancer comprising: a) administering a combination of immunotherapeutic agents including an immunotoxin comprising an antibody or antigen binding region thereof, such as a single chain variable region antibody, fused to a PE38 truncated Pseudomonas exotoxin in combination with another immunotherapeutic agents, such as immune checkpoint inhibitors, in a therapeutically effective amount to an individual having tumor, wherein the immunotherapeutic agents comprise an immunotoxin and an immune checkpoint inhibitor administered sequentially in combination therapy; b) subsequent to receiving the immunotherapeutic agents, treating the individual with anti-cancer therapy selected from the group consisting of surgery, radiation therapy, and a combination thereof, effective to reduce tumor burden (e.g., the amount of tumor) in the individual (i.e., the immunotherapeutic agents are administered before the anti-cancer therapy). The immunotherapeutic agents may further comprise addition of a pharmaceutically acceptable carrier. In one aspect, the immunotoxin comprises a single chain variable region antibody fused to an exotoxin, wherein the single chain variable region antibody has CDR1, CDR2, and CDR3 regions as shown in SEQ ID NO: 1-6 (“D2C7-IT”) or an antigen binding fragment thereof.

In this method, the individual has not previously undergone treatment to reduce the tumor burden (e.g., no treatment to reduce tumor burden). An immune checkpoint inhibitor is administered to the individual bearing tumor. An effective amount of an immunotoxin is administered to the individual, wherein the immunotoxin comprises a single chain variable region antibody which can bind EGFRwt and EGFRvIII (hence, the immunotoxin targets EGFRwt and EGFRvIII expressed on the cell surface of tumor cells) and wherein the antibody is fused to a PE38 truncated Pseudomonas exotoxin. In one aspect, the single chain variable region antibody has CDR1, CDR2, and CDR3 regions as shown in SEQ ID NO: 1-6 or an antigen binding fragment thereof. Following administration of the neoadjuvant therapy comprising immunotoxin, the individual undergoes surgery to resect the tumor, or other treatment to reduce tumor burden. Such reduction of tumor burden can occur in a time period ranging from 2 weeks to several months following neoadjuvant therapy. Optionally, the neoadjuvant therapy may further comprise administration of an immune checkpoint inhibitor to the tumor bearing individual.

According to another aspect of the invention, any one of the methods described herein may further comprise adjuvant therapy comprising administering one or more of the immunotoxin targeting EGFRwt and EGFRvIII or the immune checkpoint inhibitor to the individual following resection of the tumor. For example, following resection, an immune checkpoint inhibitor may be administered to the individual as needed in maintenance therapy. In another example, if tumor recurs following resection, immunotoxin may be administered to the individual.

According to a further aspect of the invention, provided is neoadjuvant therapy of a tumor in an individual, and use of immunotoxin targeting EGFRwt and EGFRvIII, and optionally including use of an immune checkpoint inhibitor, as a medicament or as compositions in neoadjuvant therapy of tumor, wherein the tumor bearing individual has not previously undergone a resection to treat the tumor. In one aspect, the immunotoxin comprises a single chain variable region antibody fused to a PE38 truncated Pseudomonas exotoxin, wherein the single chain variable region antibody has CDR1, CDR2, and CDR3 regions as shown in SEQ ID NO: 1-6 or an antigen binding fragment thereof; and wherein after the tumor is treated with a therapeutically effective amount of the immunotoxin targeting EGFRwt and EGFRvIII, optionally including a therapeutically effective amount of the immune checkpoint inhibitor, wherein following such treatment the tumor is then resected, or tumor burden is otherwise reduced. The neoadjuvant therapy may further comprise one or more treatments, subsequent to resection of the tumor (maintenance therapy), comprising administering a therapeutically effective amount of the immunotoxin, or a therapeutically effective amount of an immune checkpoint inhibitor, or a combination thereof.

Provided is neoadjuvant therapy of a tumor in an individual comprising administering an immunotoxin targeting EGFRwt and EGFRvIII in a therapeutically effective amount to a tumor bearing individual whose tumor has not previously undergone treatment for reducing tumor burden. The method may further comprise administering an effective amount of an immune checkpoint inhibitor to the tumor bearing individual, wherein administration is prior to treatment to reduce tumor burden. In one aspect, the immunotoxin comprises a single chain variable region antibody fused to a PE38 truncated Pseudomonas exotoxin, wherein the single chain variable region antibody has CDR1, CDR2, and CDR3 regions as shown in SEQ ID NO: 1-6 or an antigen binding fragment thereof. In another aspect, the tumor is treated with either the immunotoxin or a combination of the immunotoxin and the immune checkpoint inhibitor, and the tumor is then resected. The neoadjuvant therapy provides an improved therapeutic benefit, as compared to adjuvant therapy using an immunotoxin alone or using a combination of the immunotoxin and the immune checkpoint inhibitor. A therapeutic benefit may comprise one or more of: reduced inflammation around the site of the tumor (prior to and/or after resection); improved overall survival; improved disease-free survival; decreased likelihood of recurrence (in the primary organ and/or distant recurrence); decreased incidence of metastatic disease; and an increased antitumor immune response; or an improvement in overall objective response rate using the appropriate response assessment criteria known to those skilled in the art and depending on the type of cancer treated (e.g., for lymphoma, see Cheson et al., 2014, J. Clin. Oncology32 (27):3059-3067; for solid nonlymphoid tumors, Response Evaluation Criteria In Solid Tumors (RECIST).

These and other aspects which will be apparent to those of skill in the art upon reading the specification and provide the art with new therapeutic regimens for treating cancer.

### BRIEF DESCRIPTION OF THE DRAWINGS

FIG. 1. In vivo efficacy of D2C7-IT+(αCTLA-4 or αPD-1) mAb combination therapy in subcutaneous CT2A-D2C7 glioma-bearing C57BL/6 immunocompetent mice. FIG. 1 shows percent survival in relation to days after implantation for all the treatment groups (D2C7-IT+αCTLA-4; D2C7-IT+αPD-1; αCTLA-4; αPD-1; D2C7-IT; Vehicle Control) followed up to Day 100 (as applicable).

FIG. 2 is a graph showing tumor volume in relation to days post-implantation of tumor in mice treated with PBS and no subsequent resection of tumor (-▪-, PBS+No resection); mice treated with D2C7-IT—and with no subsequent resection of tumor (-▴-, D2C7-IT+No resection); mice treated with PBS and with subsequent resection of tumor (-▾-, PBS+Resection); and mice treated with D2C7-IT—and with subsequent resection of tumor (-♦-, D2C7-IT+Resection).

FIG. 3 shows percent survival in subcutaneous CT2A-D2C7 glioma-bearing C57BL/6 immunocompetent mice in relation to days after implantation for all the treatment groups (Vehicle Control, D2C7-IT, D2C7-IT+αPD-1, D2C7-IT+αPD-L1, D2C7-IT+αTim-3; D2C7-IT+αLag-3, and D2C7-IT+αCD73) followed up to Day 80 (as applicable).

### DETAILED DESCRIPTION OF THE INVENTION

The inventors have developed targeted immunotoxins (IT), D2C7-(scdsFv)-PE38KDEL (D2C7-IT, SEQ ID NO:5), by fusing the single chain variable fragment (scFv) from the D2C7 monoclonal antibody (mAb) with the Pseudomonas exotoxin A (PE), optionally fused to KDEL peptide. D2C7-IT reacts with both the wild-type epidermal growth factor receptor (EGFRwt) and the EGFR variant III (EGFRvIII), two proteins that are overexpressed in glioblastoma. The robust antitumor efficacy of D2C7-IT is mediated through PE in orthotopic glioma xenograft models in immunocompromised mice. In addition to direct tumor cell killing, the immunotoxin monotherapy induces a secondary antitumor immune response through the engagement of T cells. When the immunotoxin is administered in a combination regimen with an immune checkpoint inhibitor in neoadjuvant therapy, improved and synergistic results are observed.

Other moieties which can be attached to the antibodies include those which provide additional beneficial properties. For example, a KDEL (lys-asp-glu-leu) tetra-peptide can be added at the carboxy-terminus of the protein to provide retention in the endoplasmic reticulum. Variants such as DKEL, RDEL, and KNEL which function similarly can also be used. The single chain variable region antibody described herein was derived from the D2C7 monoclonal antibody. Other antibody derivatives comprising the antigen binding regions of the D2C7 antibody may also be useful in the methods described herein. These antigen binding regions or fragments of the antibody and single chain variable region antibody described herein that maintain the antigen-binding capability of the D2C7 monoclonal antibody may also be useful in the methods these include fragments of the single chain variable region antibody as well as fragments of the D2C7 antibody such as the single chain variable region antibody described herein. Other antigen binding regions may include the Fab, scFvs, and single domain or miniaturized antibodies that include less than all 6 CDRs. For use in the methods described herein, the immunotoxin comprises a single chain variable region antibody fused to a PE38 truncated Pseudomonas exotoxin. In some aspects, the single chain variable region antibody has CDR1, CDR2, and CDR3 regions as shown in SEQ ID NO: 1-6 or an antigen binding fragment thereof. By “antigen binding fragment thereof” we refer to peptides that can specifically and selectively bind to EGFRwt and EGFRvIII, comprising two or more of the CDRs which are identified as SEQ ID NO:1-6, preferably three or more of the CDRs of SEQ ID NO:1-6, alternatively four more of the CDRs of SEQ ID NO:1-6, alternatively five or more of the of the CDRs of SEQ ID NO:1-6. For example, an antigen binding fragment thereof can comprise: (a) the V_{H} chain of SEQ ID NO:7, (b) the V_{L} chain of SEQ ID NO:9, (c) both the V_{H} and V_{L} chain of SEQ ID NO:7 and 9 attached by a suitable linker, (d) V_{H} and V_{L} chain of SEQ ID NO:7 and 9 attached by a linker of SEQ ID NO:8; (e) any combination of two or more of the CDRs from the heavy and light chain selected from V_{H} CDR1 (SEQ ID NO:1), V_{H} CDR2 (SEQ ID NO:2), V_{H} CDR3 (SEQ ID NO:3) and V_{L} CDR1 (SEQ ID NO:4), V_{L} CDR2 (SEQ ID NO:5), and V_{L} CDR3 (SEQ ID NO:6) which retains its ability to specifically bind EGFRwt and EGFRvIII; (f) a peptide consecutively comprising SEQ ID Nos: 7-4, and (g) any combination thereof. Any one of the antigen binding fragments thereof can be fused to a PE38 truncated Pseudomonas exotoxin, such as the PE38KDEL (SEQ ID NO:10). One suitable example of the immunotoxin is provided in SEQ ID NO:5 (the DNA sequence encoding this immunotoxin is found in SEQ ID NO:12) or a sequence having at least 90% sequence identity to SEQ ID NO:5. By “selectively” or “specifically” we mean a single chain variable region antibody or fragment thereof is capable of binding EGFRwt and EGFRvIII (which are found on tumor cells) but does not bind other receptors found on normal cells.

Tumors which can be treated are any that react with the D2C7 antibody or an antigen binding fragment thereof. These include but are not limited to those in which at least one EGFRvIII allele is present. These may be found in breast, head and neck, brain, glioblastoma multiforme, astrocytoma, lung, or other tumors. It may be desirable to determine the presence of such an allele prior to therapy. This can be done using an oligonucleotide-based technique, such as PCR, or using an immunological technique, such as immunohistochemistry. It may be desirable to determine the amount, fraction, ratio, or percentage of cells in the tumor which express EGFR and/or EGFRvIII. The more cells which express EGFR on their surfaces, the more beneficial such antibody therapy is likely to be. Even tumors that express little to no EGFRvIII may be treated due to the ability of the antibody to bind to wild-type EGFR. Optionally, tumors may be tested prior to treatment for reactivity with D2C7 antibody. The immunotoxin itself could be used as an immunohistochemistry agent, before treatment, during treatment, or after treatment. A secondary reagent could be used with the immunotoxin for detection. It could, for example, recognize the Pseudomonas component of the immunotoxin.

Immunotoxins can be administered by any technique known in the art. Compartmental delivery may be desirable to avoid cytotoxicity for normal tissues that express EGFR. Suitable compartmental delivery methods include, but are not limited to delivery to the brain, delivery to a surgically created tumor resection cavity, delivery to a natural tumor cyst, and delivery to tumor parenchyma.

Tumors which can be treated by the method of the present invention are any which express epidermal growth factor receptor (EGFR), whether wild type, EGFRvIII, or other variants. Preferably the tumor expresses the receptor in amounts far exceeding expression by normal tissues. The mechanism of high level expression may be by genetic amplification, or other alterations, whether genetic or epigenetic. Exemplary tumors which can be treated include without limitation: malignant gliomas, breast cancer, head and neck squamous cell carcinoma, lung cancer.

Blockade of T cell immune checkpoint receptors, can be performed against any such targets, including but not limited to PD-1, PD-L1, TIM-3, LAG-3, CTLA-4, and CSF-1R and combinations of such checkpoint inhibitors. The immune checkpoint receptors may be on immune cells such as T cells, monocytes, microglia, and macrophages, without limitation. The agents which assert immune checkpoint blockade may be small chemical entities or polymers, antibodies, antibody fragments, single chain antibodies or other antibody constructs, including but not limited to bispecific antibodies and diabodies.

Immune checkpoint inhibitors which may be used according to the invention are any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, and/or anti-TIM-3 antibody. Approved checkpoint inhibitors in the U.S. include atezolizumab, ipimilumab, pembrolizumab, and nivolumab. Others in Phase 3 clinical trials include tislelizumab. The inhibitor need not be an antibody, but can be a small molecule or other polymer. If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GALS, LAG3, VISTA, KIR, 2B4, CD160, CGEN-15049, CHK1, CHK2, A2aR, and the B-7 family of ligands. Combinations of inhibitors for a single target immune checkpoint or different inhibitors for different immune checkpoints may be used. Additionally, CSF-1R blockade may be used in combination or as an alternative to immune checkpoint inhibitor(s), to ensure generation of potent and sustained immunity that effectively eliminates distant metastases and recurrent tumors. Antibodies specific for CSF-1R or drugs that inhibit or blockade CSF-1R may be used for this purpose, including but not limited to emactuzumab and AMG820. The checkpoint inhibitors are commercially available and known in the art. For example, tremelimumab, an anti-CTL4 antibody is available from MedImmune (AstraZeneca) and described in U.S. Pat. No. 6,682,736 and EP Patent No. 1141028; atezolizumab is an anti-PD-L1 available from Genentech, Inc. (Roche) and described in U.S. Pat. No. 8,217,149; ipimilumab, an anti-CTLA-4 available from Bristol-Myers Squibb Co, described in U.S. Pat. Nos. 7,605,238, 6,984,720, 5,811,097, and EP Patent No. EP1212422, among others; pembrolizumab, and anti-PD-1 antibody, available from Merck and Co and described in U.S. Pat. Nos. 8,952,136, 83,545,509, 8,900,587 and EP2170959; nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; tislelizumab available from BeiGene and described in U.S. Pat. No. 8,735,553; among others.

Examples of inhibitors of CSF-1R which may be used in the combination therapy with the immunotoxin include, without limitation, the following agents which are in clinical development: PLX3397, PLX486, RG7155, AMG820, ARRY-382, FPA008, IMC-CS4, JNJ-40346527, and MCS110. These CSF-1R inhibitors are commercially available, for example, emactuzumab (RG7155), a humanized monoclonal antibody that binds tyrosine kinase receptor colony stimulating factor 1 receptor (CSF1R) available from Genentech/Roche and described in US20110165156, U.S. Pat. Nos. 9,499,624, 9,499,626, and 9,499,625; AMG820, an anti-CSF1 monoclonal antibody available from Amgen and described in 8182813; PLX3397 (Pexidartinib, 5-[(5-Chloro-1H-pyrrolo[2,3-b]pyridin-3-yl)methyl]-N-{[6-(trifluoromethyl)-3-pyridinyl]methyl}-2-pyridinamine) and PLX7486, inhibitors of CSF-1R, and available from Plexxikon; ARRY-382, an CSF1R kinase inhibitor available from Array BioPharma Inc., FPA008 (cabiralizumab) available from Five Prime Therapeutics and described in WO2016106180; IMC-CS4, an anti-CSF1R antibody available from ImClone (an Eli Lilly subsidiary) and described in WO2011123381; JNJ-40346527, an anti-CSFR1 antibody (also known as Edicotinib, small molecule 4-Cyano-1H-imidazole-2-carboxylic acid N-(2-(4,4-dimethylcyclohex-1-enyl)-6-(2,2,6,6-tetramethyltetrahydropyran-4-yl)pyridin-3-yl)amide) available from MedKoo Biosciences, and MCS110, an anti-M-CSF monoclonal antibody, also known as lacnotuzumab, available from Novartis and described in PCT patent publication WO2007016240 A2, among others.

In a method of neoadjuvant therapy, one or more immunotherapeutic agents in a therapeutically effective amount (an immunotoxin targeting EGFRwt and EGFRvIII, or the immunotoxin and an immune checkpoint inhibitor) are administered prior to an individual undergoing reduction of tumor burden. Typically, in using two immunotherapeutic agents, the agents will be administered within days of each other. For example, an immune checkpoint inhibitor is administered followed by administration of immunotoxin at 30, 28, 21, 14, 10, 9, 8, 7, 6, 5, 4, 3, 2, or 1 day(s) after administration of the immune checkpoint inhibitor. Alternatively, it may be advantageous to administer the immunotoxin prior to administration of an immune checkpoint inhibitor, wherein the immune checkpoint inhibitor is then administered to the individual within several days after receiving the immunotoxin. Priming of a cytotoxic T lymphocyte response by the immunotoxin may take from about 5 to about 14 days. Administration of the checkpoint inhibitor may beneficially be commenced before, during, or after such priming period. Immune checkpoint inhibitors may be administered by any appropriate means known in the art for the particular inhibitor. These include intravenous, oral, intraperitoneal, sublingual, intrathecal, intracavitary, intramuscularly, and subcutaneously.

Any human tumor can be treated by this method of neoadjuvant therapy, including both pediatric and adult tumors. The tumor may be in any organ, for example, brain, prostate, breast, lung, colon, and rectum. Various types of tumors may be treated, including, for example, glioblastoma, medulloblastomas, carcinoma, adenocarcinoma, etc. Other examples of tumors include, adrenocortical carcinoma, anal cancer, appendix cancer, grade I (anaplastic) astrocytoma, grade II astrocytoma, grade III astrocytoma, grade IV astrocytoma, atypical teratoid/rhabdoid tumor of the central nervous system, basal cell carcinoma, bladder cancer, breast sarcoma, bronchial cancer, bronchoalveolar carcinoma, cervical cancer, craniopharyngioma, endometrial cancer, endometrial uterine cancer, ependymoblastoma, ependymoma, esophageal cancer, esthesioneuroblastoma, Ewing's sarcoma, extracranial germ cell tumor, extragonadal germ cell tumor, extrahepatic bile duct cancer, fibrous histiocytoma, gall bladder cancer, gastric cancer, gastrointestinal carcinoid tumor, gastrointestinal stromal tumor, gestational trophoblastic tumor, gestational trophoblastic tumor, glioma, head and neck cancer, hepatocellular cancer, Hilar cholangiocarcinoma, hypopharyngeal cancer, intraocular melanoma, islet cell tumor, Kaposi sarcoma, Langerhans cell histiocytosis, large-cell undifferentiated lung carcinoma, laryngeal cancer, lip cancer, lung adenocarcinoma, malignant fibrous histiocytoma, medulloepithelioma, melanoma, Merkel cell carcinoma, mesothelioma, endocrine neoplasia, nasal cavity cancer, nasopharyngeal cancer, neuroblastoma, oral cancer, oropharyngeal cancer, osteosarcoma, ovarian clear cell carcinoma, ovarian epithelial cancer, ovarian germ cell tumor, pancreatic cancer, papillomatosis, paranasal sinus cancer, parathyroid cancer, penile cancer, pharyngeal cancer, pineal parenchymal tumor, pineoblastoma, pituitary tumor, pleuropulmonary blastoma, renal cell cancer, respiratory tract cancer with chromosome 15 changes, retinoblastoma, rhabdomyosarcoma, salivary gland cancer, small cell lung cancer, small intestine cancer, soft tissue sarcoma, squamous cell carcinoma, squamous non-small cell lung cancer, squamous neck cancer, supratentorial primitive neuroectodermal tumor, supratentorial primitive neuroectodermal tumor, testicular cancer, throat cancer, thymic carcinoma, thymoma, thyroid cancer, cancer of the renal pelvis, urethral cancer, uterine sarcoma, vaginal cancer, vulvar cancer, and Wilms tumor. Reduction of tumor burden refers to removing (e.g., surgery or “resection”) or destroying (e.g., radiation therapy) all, or substantial amounts (debulking), of a tumor thereby reducing the amount of tumor remaining.

In addition to neoadjuvant therapy comprising administering immunotoxin targeting EGFRwt and EGFRvIII, or the immunotoxin and one or more immune checkpoint inhibitors, followed by surgical removal of the tumor or reduction of the tumor burden, treatment of the individual may comprise one or more of chemotherapy, biological therapy, and radiotherapy. These modalities may be current standard of care for treatment of certain human tumors. The neoadjuvant therapy may be administered before, during, or after the standard of care for treating tumor. For example, immunotoxin and immune checkpoint inhibitor combination comprising neoadjuvant therapy may be administered after failure of the standard of care. When a combination is specified, it may be administered separately in time as two separate agents within a single combination regimen. Alternatively, the two (or more) agents may be administered in admixture.

Immunotoxins can directly kill cancer cells that express high levels of the targeted tumor antigen. Immunotoxin monotherapy can efficiently and directly destroy tumor cells expressing targeted epitopes, such as PDPN, EGFRwt and/or its truncated variant, EGFRvIII, in xenograft malignant brain tumor models in immunocompromised mice. Immunotoxin therapy can induce a secondary anti-tumor immune response in a mouse glioma model and other tumor models, which is different from the direct killing mechanism and needs the cooperation of the immune system. Since malignant brain tumors are always a heterogeneous mass, it is possible that some tumor cells can escape from the direct targeted attack of the immunotoxin therapy due to the lack of epitopes. For this reason, the secondary anti-tumor immune response stimulated by the immunotoxin may play an important role in eliminating those tumor cells not directly targeted.

Recently, several studies successfully demonstrated that tumor regression and improved survival were achieved in murine glioma models by suppressing co-inhibitory molecules, such as CTLA4 and PD1. Based on the promising preclinical data, several clinical trials have started to investigate the utilization of immune checkpoint inhibitors to treat malignant brain tumors, either as monotherapy or combinatorial therapy with other anti-tumor agents.

However, malignant gliomas, including glioblastomas, have relatively low mutation rates, which may generate fewer and subtle tumor antigens, leading to relatively poor basal immunogenicity compared to other tumor types that respond well to immunotherapies, for example, melanoma and NSCLC. Therefore, a combination of targeted cytotoxic immunotherapy using an immunotoxin and an immune checkpoint inhibitor may provide synergistic anti-tumor effect.

A desired combinatorial therapy approach may have a lower dose of targeted cytotoxic immunotherapy comprising the immunotoxin to limit its side effects, and achieve long-term anti-tumor immunity. Immunotoxin therapy can efficiently and directly kill cancer cells that express high levels of the targeted antigen through its unique cytotoxic mechanism. Cancer cells destroyed by localized immunotoxin therapy release tumor antigens and/or other neoantigens. These antigens can then be presented by the APCs to host T cells in the local draining lymph nodes, which activate CTLs to migrate and eliminate the remaining or recurrent tumor cells expressing specific tumor antigens at the tumor site. Throughout this process, various co-inhibitory checkpoint pathways between T cells and APCs and/or between T cells and tumor cells can trigger different mechanisms to de-activate T cells, and to adjust the continuation and intensity of the anti-tumor immunity. Immune checkpoint inhibitors, such as anti-CTLA4 and anti-PD1 mAbs, can block these immunosuppressive pathways and therefore augment tumor cell death caused by lymphocytes activated by the targeted immunotoxin therapy.

While the terms used in the description of the invention are believed to be well understood by one of ordinary skill in oncology and medicine, definitions, where provided herein, are set forth to facilitate description of the invention, and to provide illustrative examples for use of the terms.

As used herein, the terms “a”, “an”, and “the” mean “one or more”, unless the singular is expressly specified (e.g., singular is expressly specified, for example, in the phrase “a single agent”).

As used herein, the term “pharmaceutically acceptable carrier” means any compound or composition or carrier medium useful in any one or more of administration, delivery, storage, stability of a composition or combination described herein. These carriers are known in the art to include, but are not limited to, a diluent, water, saline, suitable vehicle (e.g., liposome, microparticle, nanoparticle, emulsion, capsule), buffer, tracking agents, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbant, flavor agent, and the like as broadly known in the pharmaceutical art.

“Neoadjuvant therapy” is used herein to refer to anti-cancer therapy given to a tumor bearing individual before the individual undergoes surgery to remove or reduce the amount of tumor or other treatment to reduce tumor burden. Surgery can involve whole resection or partial resection of tumor. Neoadjuvant therapy may result in a reduction of tumor burden which may facilitate subsequent resection.

“Adjuvant therapy” is used herein to refer to administering cancer therapy after surgery for resection tumor or after other method of reducing tumor burden is first preformed.

“Maintenance therapy” is used herein to refer to therapeutic regimen that is given to reduce the likelihood of disease progression or recurrence. Maintenance therapy can be provided for any length of time depending on assessment of clinical parameters for assessing response to therapy.

“Survival” is used herein to refer to an individual remaining alive after treatment, and includes overall survival, and disease-free survival. Survival is typically measured by the Kaplan-Meier method. Disease-free survival refers to a treated individual remaining alive without evidence of recurrence of cancer. Overall survival refers to an individual remaining alive for a defined period of time.

The above disclosure generally describes the present invention. A more complete understanding can be obtained by reference to the following specific examples, which are provided herein for purposes of illustration only, and are not intended to limit the scope of the invention.

### Example 1

A Phase I clinical trial is conducted in individuals with tumor using immunotoxin D2C7-IT alone. The tumor was recurrent glioblastoma (GBM), and D2C7-IT alone was administered after tumor resection (adjuvant therapy). As of Apr. 11, 2018, 41 patients have been treated on the Phase I dose escalation trial of single intratumoral administration of D2C7-IT. Seventeen dose levels have been investigated (dose level 1=40 ng/mL). Dose level 17 (35,032 ng/mL) was identified as dose limiting. Additional patients are presently being enrolled on dose level 16 (23,354 ng/mL), to confirm the median tolerated dose (phase 2 dose). One patient on dose level 2 (80 ng/mL) remains disease free without additional treatment since D2C7-IT infusion, more than 32.7 months later. In addition, tumor response without additional treatment is being observed in one patient treated on dose level 10 (2,050 ng/mL) and one patient on dose level 13 (6,920 ng/mL), now more than 12.8 and 6.8 months, respectively, after treatment. Three patients, now deceased, survived for 23.7, 23.2 and 21.4 months. Two additional patients remain alive more than 18.9 and 18.2 months after intratumoral infusion of D2C7-IT.

### Example 2

Construction, expression, and purification of D2C7-(scdsFv)-PE38KDEL immunotoxin. The carboxyl terminus of the D2C7 V_{H} domain was connected to the amino terminus of the V_{L} domain by a 15-amino-acid peptide (Gly4Ser)3 linker. In order to obtain a stable IT, it is essential to ensure that during renaturation V_{H} is positioned near V_{L}. This was achieved by mutating a single key residue in each chain to cysteine, for the stabilizing disulfide bond to form. On the basis of predictions using molecular modeling and empirical data with other dsFv-recombinant ITs, we chose one amino acid in each chain to mutate to cysteine. These are residues 44 in the framework region 2 (FR2) of V_{H} and 100 in the FR4 of V_{L} (according to the Kabat numbering). Thus, we prepared an Fv that contains both a peptide linker and a disulfide bond generated by cysteine residues that replace Ser44 of V_{H} and Gly100 of V_{L}. The D2C7 (scdsFv) PCR fragment was then fused to DNA for domains II and III of Pseudomonas exotoxin A. The version of Pseudomonas exotoxin A used here, PE38KDEL, has a modified C terminus which increases its intracellular retention, in turn enhancing its cytotoxicity. The D2C7-(scdsFv)-PE38KDEL (DNA sequence SEQ ID NO:12) was expressed in E. coli under the control of T7 promoter and harvested as inclusion bodies.

### Example 3

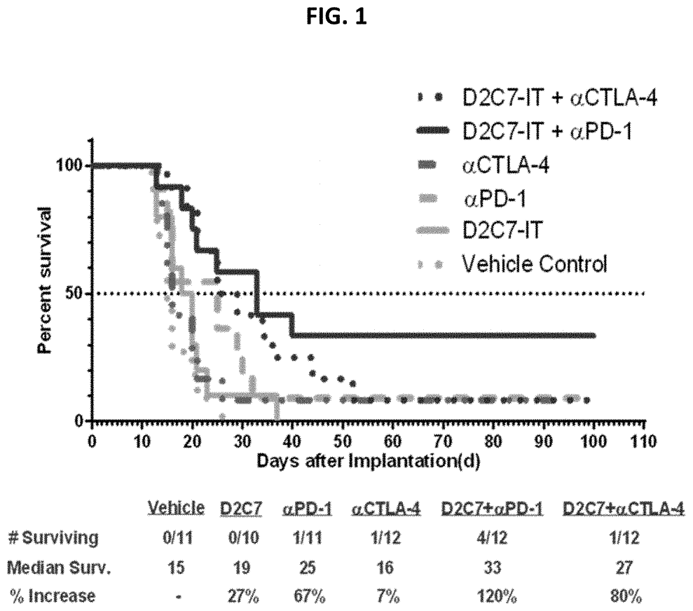

This example illustrates adjuvant therapy of cancer. Established was a mouse glioma line, CT-2A-dmEGFRvIII-Luc, overexpressing the D2C7-IT antigen mouse EGFRvIII (dmEGFRvIII). The reactivity and therapeutic efficacy of D2C7-IT against CT-2A-dmEGFRvIII-Luc (tumor cells modified to express firefly luciferase or “FFLuc”) cells was determined by flow cytometry and in vitro cytotoxicity assays, respectively. CT-2A-dmEGFRvIII-Luc were further analyzed for MHC class I and PD-L1 expression by flow cytometry. In vivo efficacy of D2C7-IT or αCTLA-4 or αPD-1 monotherapy or D2C7-IT+αCTLA-4 or D2C7-IT+αPD-1 combination therapy was evaluated in intracranial CT-2A-dmEGFRvIII-Luc glioma-bearing C57BL/6 immunocompetent mice. For this adjuvant treatment of tumor, 60 mice were randomized into 6 treatment groups (vehicle control, D2C7-IT, αPD-1, αCTLA-4, D2C7-IT+αPD-1, and D2C7-IT+αCTLA-4, 10-12 mice/group) and treated with a total dose of 0.1 μg D2C7-IT/vehicle control by convection-enhanced delivery (CED) from days 6-9. Post-implantation of CT-2A-dmEGFRvIII-Luc five doses of 250 μg/dose of rat IgG2a isotype control antibody, αPD-1 antibody, or 100 μg/dose αCTLA-4 antibody were delivered by intraperitoneal injections on days 6, 9, 12, 15, and 18. The antitumor response of intracranial (ic) tumors to treatment was assessed by the percentage increase in time to a specific neurologic endpoint (seizure activity, repetitive circling, or other subtle changes such as a decrease in appetite) or death. Animals were observed twice daily for signs of distress or development of neurologic symptoms, at which time, the mice were euthanized. Significant tumor growth delays (120% increase in median survival) and cure rate (4/12 mice) were observed in the D2C7-IT+αPD-1 combination therapy group, and significant tumor growth delay (80% increase in median survival) was observed in the D2C7-IT+αCTLA-4 combination therapy group (FIG. 1).

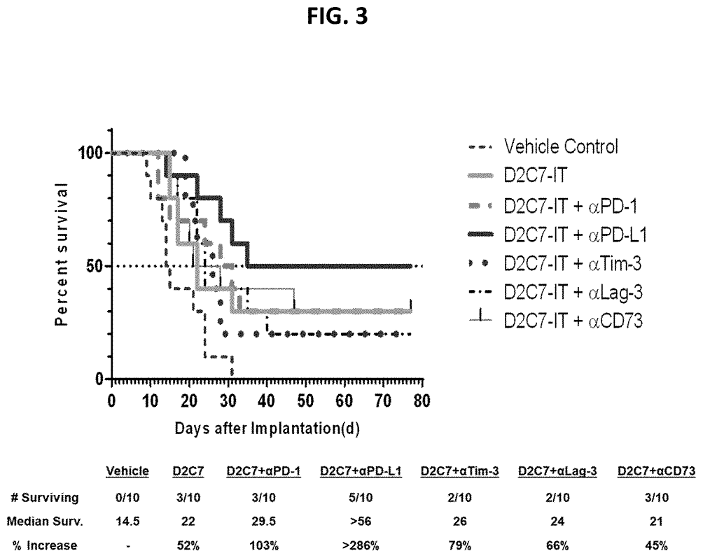

This experiment was repeated, with the following changes. D2C7-IT was used in combination therapy with additional immune checkpoint inhibitors: anti-Tim-3 antibody (FIG. 3, “αTim-3”); anti-Lag-3 antibody (FIG. 3, “αLag-3”); anti-PD-L1 antibody (FIG. 3, “αPD-L1”) and anti-CD73 antibody (FIG. 3, “αCD73”). Thus, the different combination therapies included D2C7-IT+αPD-1, D2C7-IT+αPD-L1, D2C7-IT+αTim-3; D2C7-IT+αLag-3, and D2C7-IT+αCD73. Also, dosing of with an immune checkpoint inhibitor was started prior to administration of D2C7-IT. In that regard, the immune checkpoint inhibitor was administered on days 3, 6, 9, 12, and 15; and D2C7-IT was administered from days 6-9 (as described above for D2C7-IT). As shown in FIG. 3, use of D2C7-IT and immune checkpoint inhibitor αPD-L1 in combination therapy resulted in significant increase in survival as compared to the other therapies and vehicle control.

### Example 4

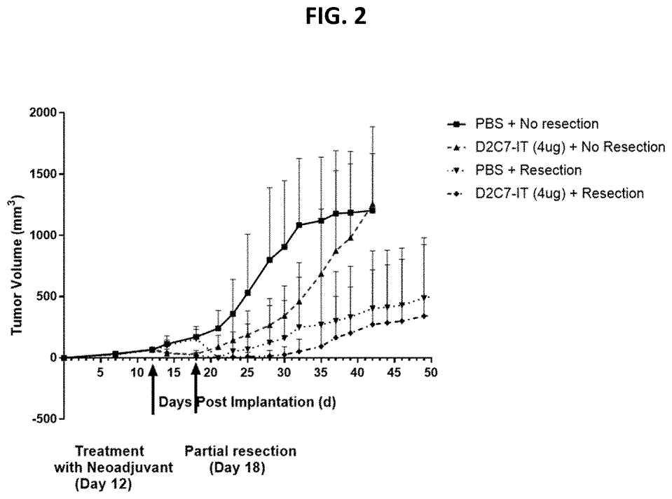

This example illustrates neoadjuvant therapy of tumor in an individual comprising administering to the individual and effective amount of an immunotoxin (e.g., D2C7-IT), after which treatment the tumor burden is then reduce in the individual. Female C57B16/J (≈20 g; 7-8 weeks) mice were injected subcutaneously in the right flank with 3×10⁶ CT2A-mEGFRVIII-D2C7-FFLuc cells suspended in 100 μls PBS. Ten mice per arm were randomly selected for vehicle or neoadjuvant (D2C7-IT) administration with surgery to reduce tumor burden or without surgery when the implanted tumors reached 50-100 mm³ The test mice were treated on Day 12 (post tumor inoculation) with a single intratumoral (i.t.) injection of 4 μg of D2C7-(scdsFv)-PE38KDEL diluted in 20 μls of PBS. The control mice were handled in the same manner and treated with 20 μls of PBS only. On Day 18 post tumor inoculation and 7 days post neoadjuvant therapy one set of control and neoadjuvant therapy mice were left untreated while a second set of control and neoadjuvant therapy mice were subjected to partial resection of tumor, with an estimated tumor volume of 0.5-1.5 mm³ remained. Tumors were measured three times a week with a handheld digital caliper and the tumor volumes were calculated in cubic millimeters by using the formula: ([length]×[width2])/2. Animals were tested out of the study when the tumor volume reached 1500-2000 mm³ or when tumor ulceration (an open sore) occurs. As shown in FIG. 2, in mice treated with PBS and no subsequent resection of tumor (-▪-, PBS+No resection) there were 0/10 mice with fully regressed tumor; in the mice treated with D2C7-IT—and with no subsequent resection of tumor (-▴-, D2C7-IT+No resection), there were 0/10 mice with fully regressed tumor; in mice treated with PBS and with subsequent resection of tumor (-▾-, PBS+Resection) there were 2/10 mice with fully regressed tumor; and in the mice treated with D2C7-IT—and with subsequent resection of tumor (-♦-, D2C7-IT+Resection), there were 5/10 mice with fully regressed tumor. Thus, a therapeutic benefit of increased tumor regression is observed when using a neoadjuvant approach to treat a tumor bearing individual. Another illustration of the neoadjuvant therapy approach in this example is to include administration of an effective amount of immune checkpoint inhibitor in combination (sequentially) with administration of an effective amount of the immunotoxin targeting EGFRwt and EGFRvIII, both administered prior to surgical resection of tumor or other method for reducing tumor burden. Given that a neoadjuvant approach using D2C7-IT alone confers a therapeutic benefit, a therapeutic benefit will be evident using a neoadjuvant approach using D2C7-IT and immune checkpoint inhibitor in combination with an immune checkpoint inhibitor (in part, because of the observed therapeutic benefit from such combination therapy).

### Example 5

Provided is a method of treating an individual having tumor, comprising administering to the individual a therapeutically effective amount of an immunotoxin targeting EGFRwt and EGFRvIII prior to surgical resection of tumor, and then performing surgery to resect the tumor from the individual. This method of neoadjuvant therapy may further comprise administering a therapeutically effective amount of an immune check point inhibitor to the individual having tumor prior to resection of tumor. For example, several days after treatment with an anti-PD-1 antibody is initiated, immunotoxin (D2C7-IT) is then administered intratumorally (for individuals with glioblastoma, infusion will be used for intratumoral administration). One-week post-administration of immunotoxin, another dose of immune checkpoint inhibitor is administered to the individual (e.g., anti-PD-1 antibody, 240 mg). Three weeks post-administration of immunotoxin, another dose of immune checkpoint inhibitor is administered to the individual (e.g., anti-PD-1 antibody, 240 mg). Four weeks post-administration of immunotoxin, the tumor is surgically resected. Five weeks post-administration of immunotoxin, and 1-week post-surgical resection, another dose of immune checkpoint inhibitor is administered to the individual (e.g., anti-PD-1 antibody, 240 mg). 7, 9, 11, and 13 weeks post-administration of immunotoxin another dose of immune checkpoint inhibitor is administered to the individual (e.g., anti-PD-1 antibody, 240 mg). Maintenance therapy comprising administering the immune checkpoint inhibiter may then proceed as medically warranted. For example, at 17, 21, 25, and every 4 weeks thereafter until ˜101 weeks post-administration of immunotoxin, immune checkpoint inhibitor may be administered (e.g., anti-PD-1 antibody may be administered every 2 weeks at 240 mg for 4 months, then every 4 weeks at 480 mg for up to 2 years.

### SEQUENCE LISTING STATEMENT

A sequence listing in text format is co-currently submitted and is incorporated by reference as part of this application. The sequence listing provides the amino acid and nucleic acid sequences for components of the immunotoxin. Specifically, the sequence listing provides: amino acid sequence of V_{H} CDR1 (SEQ ID NO:1), V_{H} CDR2 (SEQ ID NO:2), V_{H} CDR3 (SEQ ID NO:3), V_{L} CDR1 (SEQ ID NO:4), V_{L} CDR2 (SEQ ID NO:5), and V_{L} CDR3 (SEQ ID NO:6); the amino acid sequence of the variable heavy chain (V_{H}) (SEQ ID NO:7), the variable light chain (V_{L} chain) (SEQ ID NO:9), a suitable amino acid linker that links the V_{H} and V_{L} chain as in SEQ ID NO:8; the complete amino acid sequence of the immunotoxin D2C7-scdsFv-PE38KDEL (SEQ ID NO:5) along with the nucleic acid sequence (SEQ ID NO:12) encoding the entire immunotoxin D2C7-scdsFv-PE38KDEL; and the nucleic acid sequence for the D2C7-scdsFv portion (SEQ ID NO:13) and the nucleic acid sequence for the PE38KDEL portion (SEQ ID NO:14).

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of treating a tumor in a patient comprising
(a) administering to the individual a therapeutically effective amount of an immunotoxin or of an immunotoxin and immune checkpoint inhibitor, wherein the individual has not previously undergone a resection to treat the tumor,
(b) reducing the tumor burden, optionally by performing surgery to resect the tumor or treating with radiation, and
(c) administering to the individual after reducing the tumor burden in step (b) an immune check point inhibitor; wherein the immunotoxin comprises an antibody or antigen binding region thereof, a single chain variable region antibody or antigen binding fragment thereof, fused to a PE38 truncated Pseudomonas exotoxin, wherein the antigen binding region comprises at least two CDR regions selected from the group consisting of SEQ ID NO: 1-6.

2. The method of claim 1, wherein the antibody or antigen binding region thereof comprises at least four CDR regions selected from the group consisting of SEQ ID NO:1-6.

3. The method of claim 1, wherein the antibody or antigen binding region thereof comprises six CDRs of SEQ ID NO:1-6.

4. The method of claim 1, wherein the antibody or antigen binding region thereof is a single chain variable region antibody and comprises a V_{H} of SEQ ID NO:7 and a V_{L} of SEQ ID NO:9.

5. The method of claim 1, wherein the PE38 truncated Pseudomonas exotoxin is fused to a KDEL peptide.

6. The method of claim 1, wherein the antibody or antigen binding region thereof is a single chain variable region antibody and comprises SEQ ID NO:11.

7. The method of claim 1, wherein the tumor is a malignant glioma.

8. The method of claim 1, wherein the tumor is breast cancer.

9. The method of claim 1, wherein the tumor is head and neck squamous cell carcinoma or a lung cancer.

10. (canceled)

11. The method of claim 1, wherein the immunotoxin is administered directly to the tumor.

12. The method of claim 1, wherein the immune checkpoint is selected from the group consisting of PD-1, PD-L1, CTLA-4, LAG-3, TIM-3, and CSF-1R.

13. The method of claim 12, wherein the checkpoint inhibitor is an anti-PD-1 antibody.

14. The method of claim 12, wherein the checkpoint inhibitor is an anti-PD-L1 antibody.

15. The method of claim 12, wherein the checkpoint inhibitor is an anti-CTLA4 antibody.

16. The method of claim 12, wherein the checkpoint inhibitor is an anti-LAG-3 antibody.

17. The method of claim 12, wherein the checkpoint inhibitor is an anti-TIM-3 antibody.

18. The method of claim 12, wherein the checkpoint inhibitor is an anti-CSF-1R antibody.

19. The method of claim 12, wherein the checkpoint inhibitor is a small molecule inhibitor of CSF-1R.

20. The method of claim 1, wherein in step (a) the immune checkpoint inhibitor administered prior to tumor resection is administered several days before administering the immunotoxin.

21. The method of claim 1, further comprising administering multiple doses of immune checkpoint inhibitor following resection of the tumor, wherein the doses are separated by days or weeks.
